(19) United States
(12) Patent Application Publication
(10) Pub. No.: US 2024/0207098 A1
(43) Pub. Date: Jun. 27, 2024

(54) METHOD FOR PROVIDING CONTROL DATA WITH A CENTERED CORRECTION PROFILE FOR THE TREATMENT OF A CORNEA

(71) Applicant: SCHWIND EYE-TECH-SOLUTIONS GMBH, Kleinostheim (DE)

(72) Inventors: Samuel ARBA MOSQUERA, Aschaffenburg (DE); Tobias EWERING, Kleinwallstadt (DE)

(21) Appl. No.: 18/545,461

(22) Filed: Dec. 19, 2023

(30) Foreign Application Priority Data
Dec. 22, 2022 (DE) 10 2022 134 511.4

(51) Int. Cl. A61F 9/008; G16H 40/63

(52) U.S. Cl. CPC A61F 9/00804; A61F 9/00827; G16H 40/63; A61F 2009/00872; A61F 2009/00878

(57) ABSTRACT

The invention relates to a method for providing control data with a centered correction profile for the treatment of a cornea (14). The method includes ascertaining (S10) a correction profile including an optical zone and a transition zone adjoining thereto, based on a first reference center (24) from predetermined visual disorder data; determining (S12) an offset vector for the ascertained correction profile, wherein the optical zone of the correction profile is adjusted to the first reference center (24) and an optical axis of the correction profile is adjusted to a second reference center (26) by the offset vector; wherein the offset vector is composed of three vector portions; wherein the optical zone and the transition zone of the correction profile are commonly shifted by the first vector portion; wherein the optical zone is shifted within the transition zone by the second vector portion; wherein the optical axis is displaced within the optical zone by the third vector portion. Finally, control data is provided (S14), which includes the correction profile adapted by the offset vector.

### FIELD

The invention relates to a method for providing control data with a centered correction profile for the treatment of a cornea by an ophthalmological laser of a treatment apparatus. Furthermore, the invention relates to a control device, which is configured to perform the method, to a treatment apparatus with such a control device, to a computer program and to a computer-readable medium, on which the computer program is stored.

### BACKGROUND

Treatment apparatuses and methods for controlling lasers for correcting an optical visual disorder of a cornea are known in the prior art. Therein, a pulsed laser and a beam focusing device can for example be formed such that laser beam pulses effect a photodisruption or ablation in a focus situated within the tissue of the cornea to separate a lenticule from the cornea for correcting the cornea.

For planning the correction, correction profiles are usually determined with the aid of preceding diagnostic measurements. For example, a corneal topography can be created among other things, wherein the correction profile, that is the volume body to be removed, is adjusted based on the topography. Alternatively, the correction profile can for example be planned with the aid of wavefront measurements, wherein here, since the wavefronts are measured through the pupil, the pupil center serves as a reference for adjusting the correction profile. In case of an asymmetric or decentered cornea, there is disagreement if the correction profile is to be adjusted to a pupil center or a corneal vertex. Therein, an incorrectly placed correction profile can result in an under-correction and other undesired side effects, such as for example higher order aberrations.

### SUMMARY

Therefore, the object of the invention is in improving a centering of a correction profile, in particular for an asymmetric eye.

This object is solved by the examples herein. Advantageous developments of the invention are specified in the respective dependent claims, in the following description as well as the figures.

The invention is based on the idea that a mixture of multiple centering strategies is used, that is of the adjustment to the corneal vertex and to the pupil center, to consider all of the effects, which occur in the respective reference center.

By the invention, a method for providing control data with a centered correction profile for the treatment of a cornea, in particular an asymmetric and/or decentered cornea, by an ophthalmological laser of a treatment apparatus is provided, wherein the method is performed by a control device. Preferably, the method can be applied for a cornea, in which an asymmetry of the corneal vertex with the pupil center is present, which means that they are not situated on a visual or optical axis.

As the steps, the method includes ascertaining a correction profile including an optical zone and a transition zone adjoining thereto, based on a first reference center from predetermined visual disorder data. Herein, the reference center can be the pupil center, the corneal vertex, a corneal apex (point of maximum curvature of the cornea), a point of minimum thickness of the cornea, a video keratoscope axis and/or a visual/optical axis. Therein, the correction profile, which can for example be determined for an ablation and/or photodisruption, as usual, comprises an optical zone, by which a correction is provided, and a transition zone adjoining thereto at the edge of the optical zone, at which a uniform or gentle transition of the optical zone into the residual tissue of the cornea is provided.

Subsequently, determining an offset vector for the ascertained correction profile is effected, wherein the optical zone of the correction profile is adjusted to the first reference center and an optical axis of the correction profile is adjusted to a second reference center, in particular to a corneal vertex, by the offset vector. Herein, the second reference center can also be the pupil center, the corneal vertex, a corneal apex, a point of minimum thickness of the cornea, a video keratoscope axis, and/or a visual/optical axis, wherein the first and the second reference center differ. Thus, the first reference center is an original center, which was selected due to an initial planning of the correction. The second reference center is a target center, which is also to be taken into account in the correction. The offset vector is composed of three vector portions, wherein the optical zone and the transition zone of the correction profile are commonly shifted by the first vector portion, wherein the optical zone is shifted within the transition zone by the second vector portion, and wherein the optical axis is displaced within the optical zone by the third vector portion.

In other words, the correction profile previously ascertained based on the first reference center is adapted by means of an offset vector. By the offset vector, it is to be achieved that the optical zone is adjusted to the reference center, thus for example concentrically to the pupil center. Furthermore, it is to be achieved by the offset vector that the optical axis of the correction profile is adjusted to the second reference center, in particular the corneal vertex. Thus, the correction profile can be asymmetrically adapted and preferably compensate for multiple effects, which otherwise occur upon adjustment to only one of the reference centers. Therein, the offset vector is determined from three different approaches, which are taken into account in respective vector portions of the offset vector, in particular via an addition of the three vector portions.

Theren, the complete correction profile, thus the optical zone together with the transition zone, is shifted within the cornea in the first vector portion. In the second vector portion, only the optical zone is adapted in that it is offset within the transition zone, which defines the outer circumference of the correction profile. By the third vector portion, the optical axis of the correction profile or of the optical zone is changed. Preferably, each of the three vector portions can be greater than 0 for the offset vector. Alternatively, one of the vector portions can also be 0 and only two of the vector portions are taken into account.

In a final step, providing the control data for the ophthalmological laser of the treatment apparatus is effected, which includes the correction profile adapted by the offset vector. The treatment apparatus and/or the ophthalmological laser can subsequently be controlled by the control data for correcting a visual disorder of the cornea.

By the invention, the advantage arises that improved correction profiles can be achieved, by which less side effects such as for example an introduction of asymmetric aberrations, in particular coma and trefoil, can be achieved.

The invention also includes developments, by which additional advantages arise.

A development provides that the entire correction profile is shifted from the first reference center towards the second reference center, in particular towards an optical axis of the eye or the corneal vertex, by the first vector portion, wherein corneal and/or ocular wavefront measurements are converted to new positions based on the first vector portion. In other words, the shift is effected by the first vector portion from the first reference center, for example from the pupil center, towards an optical axis, which can for example be defined by the corneal vertex. Therein, wavefront measurements of the cornea and/or of the entire eye additionally performed for the treatment can preferably also be adapted or converted, wherein a statement about higher order aberrations thus arises, which can be taken into account for the correction. By this development, a preferred form of configuration for determining the first vector portion is achieved.

A further development provides that the transition zone is kept concentrical to the first reference center and the optical zone is shifted towards the second reference center, in particular towards the corneal vertex, by the second vector portion. This means that the transition zone, thus the diameter of the correction profile, is adjusted concentrically to the first reference center and only the optical zone is shifted to the second reference center, which is preferably the corneal vertex, within the correction profile. By this development, a preferred form of configuration of the second vector portion can be achieved.

A further development provides that the correction profile is asymmetrically deformed for displacing the optical axis by the third vector portion.

Preferably, it can be provided that a temporary correction profile with an increased optical zone is calculated for asymmetrically deforming the correction profile, wherein the increased optical zone is the original optical zone added with a double magnitude of the third vector portion. Subsequently, the temporary correction profile is shifted by the single magnitude of the third vector portion in opposite direction and scaled back into the original optical zone. In other words, a radius of curvature of the correction profile can be adapted by means of the third vector portion to displace the optical axis. A possibility of implementing this is generating a temporary correction profile, in which it applies:

$$O Z = O {Z}_{nominal} + 2 * Offset$$

wherein OZ is the increased optical zone, OZₙₒₘᵢₙₐₗ is the optical zone originally determined for the correction profile and the Offset is the magnitude of the third vector portion. This temporary correction profile can then be shifted back in opposite direction of the third vector portion, namely with the magnitude of the third vector portion, wherein the temporary correction profile is subsequently scaled back to the size of the original optical zone. Accordingly, an asymmetric or aspheric correction profile is provided. In other words, the asymmetric deformation can serve to cover the pupil edges and be centered to the corneal vertex at the same time.

Particularly preferably, it is provided that inclination components are additionally removed in the asymmetrically deformed correction profile. This means that tilt components (C[1,+/−1]=0) can preferably be compensated for by means of known methods in the deformed correction profile to compensate for additional effects deteriorating the correction.

Overall, a preferred configuration of the third vector portion can thus be achieved.

A further development provides that the three vector portions form the offset vector according to respectively preset portion factors. This means that the offset vector does not have to be formed uniformly, in particular each to a third, by the vector portions, wherein this is preferably provided, but that they have a respective portion factor, which specifies how severely the respective vector portion acts on the offset vector.

Particularly advantageously, it is provided that one of the following portion factors is preset:
The portion factor of only one vector portion is 100 percent of the offset vector, or
the portion factor of two vector portions is 50 percent of the offset vector, or
the portion factor for each vector portion is one third of the offset vector, or
the portion factor of two vector portions is 25 percent and of one vector portion is 50 percent of the offset vector.

Thus, a suitable compensation for the correction profile in case of asymmetric corneas can be achieved from a mixture of the offset vectors and the various processes described thereby. Preferably, the portion factor can be preset. For example, the vector portions with the portion factor can be ascertained by solving a minimization problem to adapt the correction profile.

The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

Furthermore, a method for controlling a treatment apparatus is provided. Therein, the method includes the method steps of at least one embodiment of a method as it was previously described. Furthermore, the method for controlling the treatment apparatus also includes the step of transferring the provided control data to at least one ophthalmological laser of the treatment apparatus.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by means of optical breakthrough, in particular at least partially separate it by means of photodisruption and/or to ablate corneal layers by means of (photo)ablation.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the developments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 a schematic representation of a treatment apparatus according to an exemplary embodiment.

FIG. 2 a schematic method diagram for centering a correction profile according to an exemplary embodiment.

In the figures, identical or functionally identical elements are provided with the same reference characters.

### DETAILED DESCRIPTION

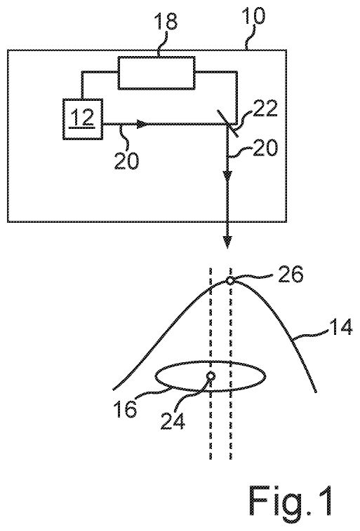

The FIG. 1 shows a schematic representation of a treatment apparatus 10 with an eye surgical laser 12 for the correction of a cornea 14, wherein a correction profile, in particular a volume body or a lenticule (not shown), can for example be defined by control data, which can be separated from the cornea 14 by means of photodisruption and/or ablation. For separating the lenticule, interfaces can for example be preset in the control data, on which a cavitation bubble path for separating the lenticule from the cornea 14 can be generated. Therein, the correction profile usually includes an optical zone, in which the correction is to occur, and a transition zone adjoining thereto, in which the interfaces for example converge such that a gentle transition into the residual tissue can be achieved.

One recognizes that a control device 18 for the laser 12 can be formed besides the laser 12, such that it can emit pulsed laser pulses for example in a predefined pattern for generating the correction profile or the interfaces. Alternatively, the control device 18 can be a control device 18 external with respect to the treatment apparatus 10.

Furthermore, the FIG. 1 shows that the laser beam 20 generated by the laser 12 is deflected towards the cornea 14 by means of a beam device 22, namely a beam deflection device such as for example a rotation scanner. The beam deflection device 22 is also controlled by the control device 18 to generate the correction profile or the interfaces.

Preferably, the illustrated laser 12 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 KHz and 100 MHz. In addition, the control device 18 optionally comprises a storage device (not illustrated) for at least temporary storage of at least one control dataset, wherein the control dataset or datasets include(s) control data for positioning and/or for focusing individual laser pulses in the cornea. The position data and/or the focusing data of the individual laser pulses, that is the correction profile of the lenticule to be separated, are generated based on predetermined control data, in particular from previously measured visual disorder data, in particular a previously measured topography and/or pachymetry and/or the morphology of the cornea.

For determining the visual disorder data, which can for example indicate a value in diopters, suitable examination data for describing the visual disorder can be received by the control device 18 from a data server or the examination data can be directly input into the control device 18.

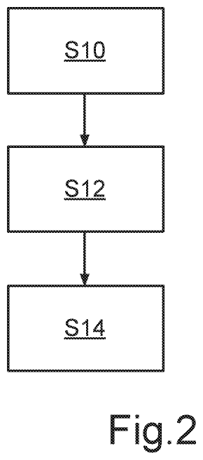

In case of a deformed or asymmetric cornea 14, as it is illustrated in FIG. 1, it can occur that the pupil center 24 of the pupil 16 does not coincide with a visual axis, which can be related to a corneal vertex 26. Thus, in planning the correction profile, according to used method for planning, an offset between the pupil center 24 and the corneal vertex 26 is present. If the correction profile is for example planned by means of wavefront measurements by means of an aberrometer, the pupil center 24 serves as the reference center, since the wavefronts are measured through the pupil 16. In contrast, if the correction profile is for example planned based on a topography, the corneal vertex 26 usually serves as the reference center, since it specifies the highest point of the topography. In order to consider this discrepancy in planning the correction profile, the control device 18 can be formed to perform the method schematically illustrated in FIG. 2 for compensating for the asymmetry.

In a step S10, the correction profile can be ascertained based on a first reference center, wherein the pupil center 24 is here for example present as the first reference center. For example, the correction profile can be ascertained with the aid of wavefront measurements, which determine the wavefronts through the pupil 16. In this correction profile, which has been ascertained based on the pupil center 24, however, in case of asymmetric eyes, an offset to a second reference center, for example to the visual axis of the eye, can be present, which can be adjusted to the corneal vertex 26. The corneal vertex 26 can for example be ascertained by means of topography measurements. In order to compensate for this offset and to consider both reference centers 24, 26, it can be provided that an offset vector is determined for the ascertained correction profile in a step S12.

By the offset vector, it is to be achieved that an optical zone of the correction profile is adjusted to the first reference center, thus to the pupil center 24, and an optical axis of the correction profile is adjusted to the second reference center, thus to the corneal vertex 26. This means, the correction profile is to be shifted in the cornea 14 by the offset vector such that it is situated above the pupil 16, but the optical axis coincides with the visual axis, which is set by the corneal vertex 26.

Thereto, the offset vector is composed of three vector portions, which each have an influence on the shift and/or the deformation of the correction profile.

For example, the entire correction profile can first be shifted by a first vector portion, in particular from the pupil center 24 towards a projection of the corneal vertex 26. This means that the optical zone and the transition zone are offset at the same time. Herein, the wavefront measurements, which are adjusted based on the pupil center 24, can preferably also be adapted corresponding to the first vector portion, to additionally consider higher order aberrations.

Subsequently, the optical zone can for example be shifted within the transition zone by a second vector portion, such that it is for example no longer surrounded by the transition zone in centered manner. By the second vector portion, the transition zone can be preferably kept concentrical to the pupil center 24 and only the optical zone of the correction profile is shifted towards the corneal vertex 26, wherein the optical zone therein does not have to concentrically coincide with the corneal vertex 26.

By a third vector portion, the optical axis of the correction profile is then displaced within the optical zone, which means that the correction profile is asymmetrically deformed. For implementing the asymmetric deformation of the correction profile, it can be provided as a simple and preferred form of configuration that a temporary correction profile with an increased optical zone is calculated, wherein the original optical zone is extended to one side with the two-fold value of the third vector portion. Subsequently, the increased temporary correction profile can be shifted back in opposite direction to the third vector portion and be scaled back into the original optical zone. Herein, inclination components can preferably also be removed in the thus obtained correction profile.

The respective vector portions can additionally be multiplied or divided by portion factors to control an influence of the respective vector portions on the offset vector. Herein, multiple distributions of the portion factor can be provided, wherein a sum of the portion factors preferably results in 1.

For example, it can be provided that the portion factor of a vector portion constitutes 100 percent of the offset vector and thus the respectively other vector portions are not taken into account. In a further example, it can be provided that the portion factor of two vector portions constitutes each half of the offset vector, which means that only two vector portions of the offset vector are taken into account. In a particularly preferred example, it can be provided that the portion factor of each vector portion is one third of the offset vector. Thus, all of the previously presented processes for compensating for the correction profile are uniformly considered. Alternatively, it can further be provided that a vector portion is more severely considered by the portion factor, in particular to 50 percent, and the remaining two vector portions obtain a respective portion factor of one quarter.

Finally, the correction profile can be adapted by the offset vector and the correction profile adapted by the offset vector can be provided by means of control data in a step S14. Then, the control data can be used by the control device 18 to control the laser 12 of the treatment apparatus 10 for correcting a visual disorder.

Overall, the examples show, how an asymmetric centering strategy for a refractive treatment procedure can be achieved by combination of information of the pupil 16 and of the corneal vertex 26.

1. A method for providing control data with a centered correction profile for treatment of a cornea of an eye by an ophthalmological laser of a treatment apparatus, wherein the method comprises the following steps performed by a control device:
ascertaining a correction profile including an optical zone and a transition zone adjoining thereto, based on a first reference center from predetermined visual disorder data;
determining an offset vector for the ascertained correction profile, wherein the optical zone of the correction profile is adjusted to the first reference center and an optical axis of the correction profile is adjusted to a second reference center by the offset vector:
wherein the offset vector is composed of three vector portions;
wherein the optical zone and the transition zone of the correction profile are commonly shifted by the first vector portion;
wherein the optical zone is shifted within the transition zone by the second vector portion;
wherein the optical axis is displaced within the optical zone by the third vector portion;
providing the control data for the ophthalmological laser of the treatment apparatus, which includes the correction profile adapted by the offset vector.

2. The method according to claim 1, wherein the correction profile is shifted from the first reference center towards the second reference center, in particular towards an optical axis of the eye or a corneal vertex, by the first vector portion, wherein corneal and/or ocular wavefront measurements are converted to new positions based on the first vector portion.

3. The method according to claim 1, wherein the transition zone is kept concentric to the first reference center and the optical zone is shifted towards the second reference center by the second vector portion.

4. The method according to claim 1, wherein the correction profile is asymmetrically deformed for displacing the optical axis by the third vector portion.

5. The method according to claim 4, wherein a temporary correction profile with an increased optical zone is calculated for asymmetrically deforming the correction profile, wherein the increased optical zone is an original optical zone added with a double magnitude of the third vector portion, wherein the temporary correction profile is shifted by a single magnitude of the third vector portion in opposite direction and is scaled back into the original optical zone.

6. The method according to claim 5, wherein inclination components are additionally removed in the asymmetrically deformed correction profile.

7. The method according to claim 1, wherein the three vector portions form the offset vector according to respectively preset portion factors.

8. The method according to claim 7, wherein one of the following portion factors is preset:
a portion factor of only one vector portion is 100% of the offset vector; or
a portion factor of two vector portions is 50% of the offset vector; or
a portion factor for each vector portion is one third of the offset vector; or
a portion factor of two vector portions is 25% and of one vector portion is 50% of the offset vector.

9. A control device, which is configured to perform a respective method according to claim 1.

10. A treatment apparatus with at least one eye surgical laser for separation of a corneal volume with predefined interfaces of a human or animal eye by means of photoablation and/or photodisruption, and at least one control device according to claim 9.

11. A non-transitory computer readable medium configured for storing a computer program, the computer program including commands which cause a treatment apparatus to execute the method according to claim 1.

12. (canceled)
